(19) United States
(12) Patent Application Publication
(10) Pub. No.: US 2004/0162542 A1
(43) Pub. Date: Aug. 19, 2004

(54) Methods and systems for treating the vasculature with estrogens

(75) Inventors: Judith Carol Wilber, Oakland, CA (US); Lynn Mateel Barr, Lafayette, CA (US); Kirk Patrick Seward, Dublin, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: EndoBionics, INc., San Leandro, CA

(21) Appl. No.: 10/728,186

(22) Filed: Dec. 3, 2003

Related U.S. Application Data
(60) Provisional application No. 60/430,993, filed on Dec. 3, 2002.

(51) Int. Cl. A61M 31/00

(52) U.S. Cl. 604/507

(57) ABSTRACT

Methods and systems for inhibiting neointimal hyperplasia and treating vulnerable plaque comprise direct needle injection of estrogen into a perivascular space or adventitial tissue.

### CROSS-REFERENCES TO RELATED APPLICATIONS

The present application is claiming the benefit under 35 USC 119(e) of U.S. Provisional Application No. 60/430,993 (Attorney Docket No. 21621-001300US), filed on Dec. 3, 2002, the full disclosure of which is incorporated herein by reference.

### BACKGROUND OF THE INVENTION

1. Field of the Invention

The present invention relates generally to medical methods and devices. More particularly, the present invention relates to methods and systems for injecting estrogens into the perivascular and/or adventitial regions surrounding a blood vessel.

One drug which has been proposed for inhibiting vascular hyperplasia following stenting or other arterial interventions is 17-beta-estradiol. This estrogen drug has been experimentally delivered to animal models using a sleeve catheter and shown to decrease neointimal hyperplasia after balloon angioplasty in pigs. While such experiments have been promising, local delivery of the drug using a sleeve catheter limits the uptake of the drug through the endothelium and to the adventitia. Thus, presently proposed delivery protocols result in inefficient uptake of the drug through the endothelium and into the adventitia surrounding the arterial wall. The limited concentrations of the estradiol which are likely achieved within the adventitia almost certainly limit the effectiveness of the drug in inhibiting hyperplasia.

For these reasons, it would be desirable to provide alternative and improved methods and systems for delivering estradiols and other estrogens to target locations within arteries and other blood vessels. Such methods and systems are preferably effective at treating both neointimal hyperplasia and vulnerable plaque.

2. Description of the Background Art

The local delivery of 17-beta-estradiol to coronary arteries injured by balloon angioplasty in pig models is disclosed in Chandrasekar et al. (2001) J. Am. Col. Cardiol., 38:1570 and (2000) J. Am. Col. CardioL, 36:1972. Delivery was performed using a sleeve catheter positioned over a balloon catheter. In vitro inhibition of proliferation of human vascular smooth muscle cells is described in Espinosa et al. (1996) Cardiovasc. Res., 32:980-85. WO 01/21157 describes the local delivery of 17-beta-estradiol for preventing vascular intimal hyperplasia and improving endothelium function after vascular injury. Local delivery was accomplished using the Infusesleeve™ sleeve delivery catheter, LocalMed, Palo Alto, Calif. The Infusesleeve™ catheter is described generally in U.S. Pat. No. 5,336,178.

### BRIEF SUMMARY OF THE INVENTION

Methods and systems according to the present invention provide for the injection of an estrogen into tissue surrounding a body lumen, typically into the perivascular tissue surrounding a blood vessel, usually an artery, and more usually a coronary artery. The estrogen is injected into a location beyond the endothelium of the blood vessel, typically by a distance equal to at least 10% of the mean luminal diameter of the blood vessel at the site of injection, more typically being in the range from 10% to 50% of the mean luminal diameter. The estrogen is typically an estradiol, more typically being selected from the group consisting of 17-beta- estradiol and estradiol cypionate.

The methods of the present invention are useful for treating arteries and other blood vessels which are at risk of hyperplasia, such as neointimal hyperplasia following balloon angioplasty, stenting, or other primary interventional treatment for arteriosclerotic disease. Alternatively, the methods of the present invention may be useful for the primary treatment of vulnerable plaque. In both cases, the estrogen may be injected at a location proximate to the diseased region. Alternatively, the estrogen may be injected at a location remote from the location of the disease, where the estrogen will migrate to the diseased region and elsewhere through the adventitia. In some instances, the estrogen may be injected in adjacent arteries or even veins, where the estrogen will permeate through the adventitia over the blood vessel of interest and optionally adjacent blood vessels.

The estrogen will be injected using an intravascular catheter, typically by advancing a needle from the catheter to a location past the endothelial layer of the blood cell and into a perivascular space or directly into the adventitia. Usually, a sufficient amount of the estrogen will be injected to circumferentially permeate around the blood vessel and endothelium over an axial length of at least 1 cm, preferably at least 2 cm, and often times 3 cm, or greater.

The present invention further provides systems comprising an amount of an estrogen in combination with an intravascular catheter having a needle for injecting the estrogen into a location beyond the endothelium of a blood vessel. A system may further comprise instructions for use according to any of the methods described above.

### BRIEF DESCRIPTION OF THE DRAWINGS

FIG. 1A is a schematic, perspective view of a microfabricated surgical device for interventional procedures in an unactuated condition.

FIG. 1B is a schematic view along line 1B-1B of FIG. 1A.

FIG. 1C is a schematic view along line 1C-1c of FIG. 1A.

FIG. 2A is a schematic, perspective view of a microfabricated surgical device for interventional procedures in an actuated condition.

FIG. 2B is a schematic view along line 2B-2B of FIG. 2A.

FIG. 3 is a schematic, perspective view of the microfabricated surgical device of the present invention inserted into a patient's vasculature.

FIGS. 4A-4G are schematic, perspective views illustrating steps in the fabrication of a microfabricated surgical device of the present invention.

FIG. 5 is a schematic, perspective view of another embodiment of the device of the present invention.

FIG. 6 is a schematic, perspective view of still another embodiment of the present invention, as inserted into a patient's vasculature.

FIGS. 7A and 7B are schematic views of other embodiments of the device of the present invention (in an unactuated condition) including multiple needles.

FIG. 8 is a schematic view of yet another embodiment of the device of the present invention (in an unactuated condition).

### DETAILED DESCRIPTION OF THE INVENTION

The present invention provides methods and systems for the direct injection of an estrogen into a perivascular space or into adventitial tissue surrounding the endothelial layer of the blood vessel. Because the estrogens are being injected into the tissue of interest, rather than into the endothelial layer, generally lower doses of the drug may be used than have been suggested in the prior art, such as in WO 01/21157. In particular, it is believed that the direct injection methods of the present invention may successfully utilize dosages of 17-beta-estradiol below 200 μg, sometimes below 100 μg, and sometimes below 50 μg, per injection. Of course, because of the ability of the present invention to achieve adventitial distribution of the drug, in many instances doses above 200 μg may be useful. Thus, doses of 400 μg, 600 μg, and higher may also find use in the present invention.

The present invention will preferably utilize microfabricated devices and methods for intravascular injection of the estrogens. The following description provides several representative embodiments and processes for fabricating a microfabricated needle or microneedle, or even a macroneedle, for the delivery of the estrogens into a perivascular space or adventitial tissue. The perivascular space is the potential space between the outer surface and the endothelium or “vascular wall” of either an artery or vein. The microneedle is inserted substantially normal to the wall of a vessel (artery or vein) to eliminate as much trauma to the patient as possible. Until the microneedle is at the site of an injection, it is positioned out of the way so that it does not scrape against arterial or venous walls with its tip. Specifically, the microneedle remains enclosed in the walls of an actuator or sheath attached to a catheter so that it will not injure the patient during intervention or the physician during handling. When the injection site is reached, movement of the actuator along the vessel terminated, and the actuator is operated to cause the microneedle to be thrust outwardly, substantially perpendicular to the central axis of a vessel, for instance, in which the catheter has been inserted.

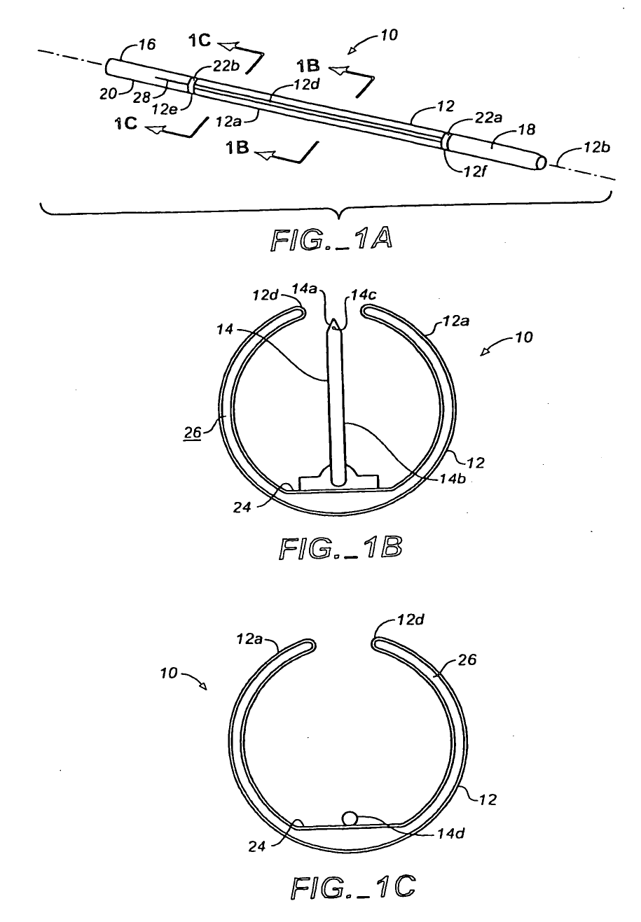

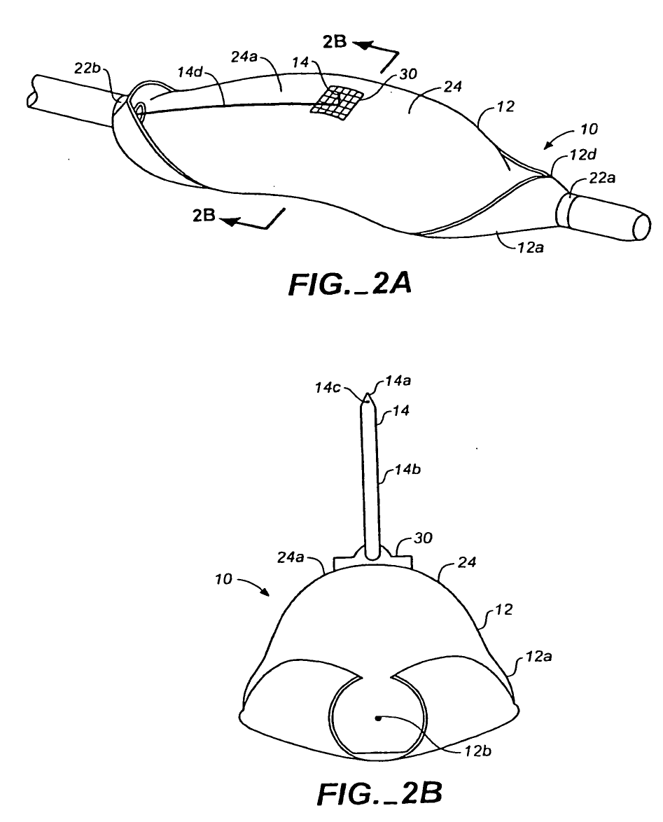

As shown in FIGS. 1A-2B, a microfabricated intravascular catheter 10 includes an actuator 12 having an actuator body 12a and central longitudinal axis 12b. The actuator body more or less forms a C-shaped outline having an opening or slit 12d extending substantially along its length. A microneedle 14 is located within the actuator body, as discussed in more detail below, when the actuator is in its unactuated condition (furled state) (FIG. 1B). The microneedle is moved outside the actuator body when the actuator is operated to be in its actuated condition (unfurled state) (FIG. 2B).

The actuator may be capped at its proximal end 12e and distal end 12f by a lead end 16 and a tip end 18, respectively, of a therapeutic catheter 20. The catheter tip end serves as a means of locating the actuator inside a blood vessel by use of a radio opaque coatings or markers. The catheter tip also forms a seal at the distal end 12f of the actuator. The lead end of the catheter provides the necessary interconnects (fluidic, mechanical, electrical or optical) at the proximal end 12e of the actuator.

Retaining rings 22a and 22b are located at the distal and proximal ends, respectively, of the actuator. The catheter tip is joined to the retaining ring 22a, while the catheter lead is joined to retaining ring 22b. The retaining rings are made of a thin, on the order of 10 to 100 microns (μm), substantially rigid material, such as Parylene (types C, D or N), or a metal, for example, aluminum, stainless steel, gold, titanium or tungsten. The retaining rings form a rigid substantially “C”-shaped structure at each end of the actuator. The catheter may be joined to the retaining rings by, for example, a butt-weld, an ultra sonic weld, integral polymer encapsulation or an adhesive such as an epoxy.

The actuator body further comprises a central, expandable section 24 located between retaining rings 22a and 22b. The expandable section 24 includes an interior open area 26 for rapid expansion when an activating fluid is supplied to that area. The central section 24 is made of a thin, semi-rigid or rigid, expandable material, such as a polymer, for instance, Parylene (types C, D or N), silicone, polyurethane or polyimide. The central section 24, upon actuation, is expandable somewhat like a balloon-device.

The central section is capable of withstanding pressures of up to about 100 atmospheres upon application of the activating fluid to the open area 26. The material from which the central section is made of is rigid or semi-rigid in that the central section returns substantially to its original configuration and orientation (the unactuated condition) when the activating fluid is removed from the open area 26. Thus, in this sense, the central section is very much unlike a balloon which has no inherently stable structure.

The open area 26 of the actuator is connected to a delivery conduit, tube or fluid pathway 28 that extends from the catheter's lead end to the actuator's proximal end. The activating fluid is supplied to the open area via the delivery tube. The delivery tube may be constructed of Teflon© or other inert plastics. The activating fluid may be a saline solution or a radio-opaque dye.

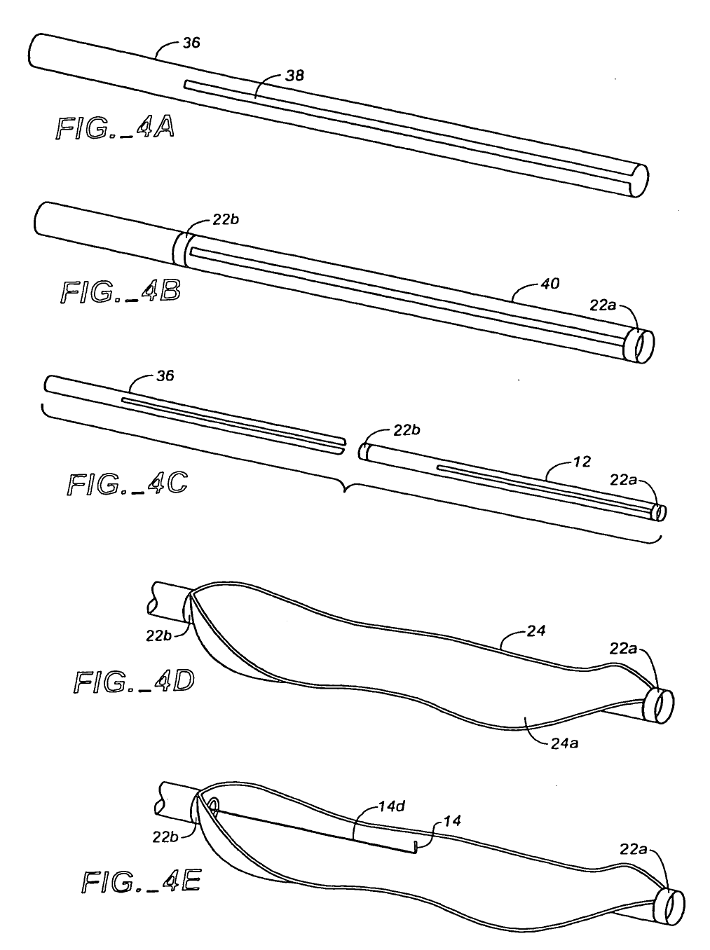

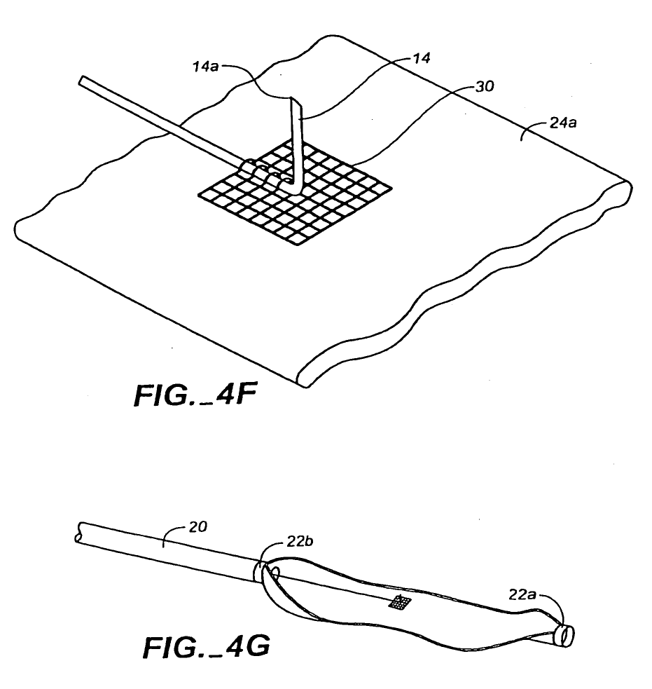

The microneedle 14 may be located approximately in the middle of the central section 24. However, as discussed below, this is not necessary, especially when multiple microneedles are used. The microneedle is affixed to an exterior surface 24a of the central section. The microneedle is affixed to the surface 24a by an adhesive, such as cyanoacrylate. Alternatively, the microneedle maybe joined to the surface 24a by a metallic or polymer mesh-like structure 30 (See FIG. 4F), which is itself affixed to the surface 24a by an adhesive. The mesh-like structure may be-made of, for instance, steel or nylon.

The microneedle includes a sharp tip 14a and a shaft 14b. The microneedle tip can provide an insertion edge or point. The shaft 14b can be hollow and the tip can have an outlet port 14c, permitting the injection of a pharmaceutical or drug into a patient. The microneedle, however, does not need to be hollow, as it may be configured like a neural probe to accomplish other tasks.

As shown, the microneedle extends approximately perpendicularly from surface 24a. Thus, as described, the microneedle will move substantially perpendicularly to an axis of a vessel or artery into which has been inserted, to allow direct puncture or breach of vascular walls.

The microneedle further includes a pharmaceutical or drug supply conduit, tube or fluid pathway 14d which places the microneedle in fluid communication with the appropriate fluid interconnect at the catheter lead end. This supply tube may be formed integrally with the shaft 14b, or it may be formed as a separate piece that is later joined to the shaft by, for example, an adhesive such as an epoxy.

The needle 14 may be a 30-gauge, or smaller, steel needle. Alternatively, the microneedle may be microfabricated from polymers, other metals, metal alloys or semiconductor materials. The needle, for example, may be made of Parylene, silicon or glass. Microneedles and methods of fabrication are described in U.S. application Ser. No. 09/877,653, filed Jun. 8, 2001, entitled “Microfabricated Surgical Device”, assigned to the assignee of the subject application, the entire disclosure of which is incorporated herein by reference.

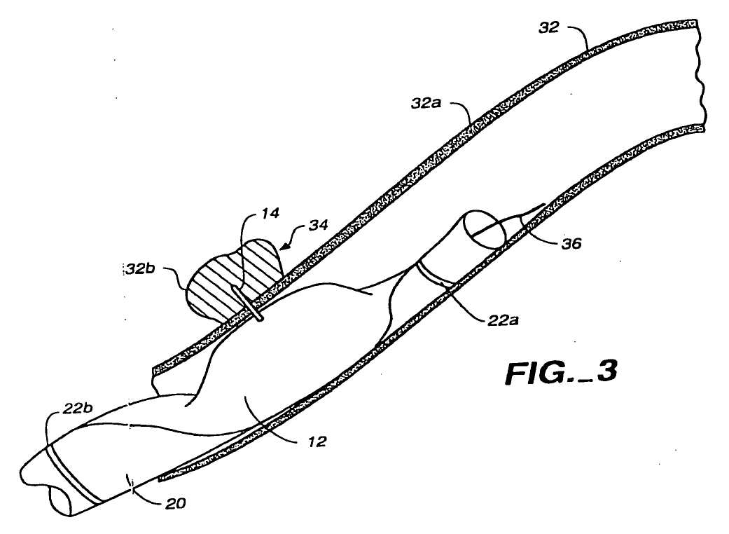

The catheter 20, in use, is inserted through an artery or vein and moved within a patient's vasculature, for instance, a vein 32, until a specific, targeted region 34 is reaches (see FIG. 3). As is well known in catheter-based interventional procedures, the catheter 20 may follow a guide wire 36 that has previously been inserted into the patient. Optionally, the catheter 20 may also follow the path of a previously-inserted guide catheter (not shown) that encompasses the guide wire.

During maneuvering of the catheter 20, well-known methods of fluoroscopy or magnetic resonance imaging (MRI) can be used to image the catheter and assist in positioning the actuator 12 and the microneedle 14 at the target region. As the catheter is guided inside the patient's body, the microneedle remains unfurled or held inside the actuator body so that no trauma is caused to the vascular walls.

After being positioned at the target region 34, movement of the catheter is terminated and the activating fluid is supplied to the open area 26 of the actuator, causing the expandable section 24 to rapidly unfurl, moving the microneedle 14 in a substantially perpendicular direction, relative to the longitudinal central axis 12b of the actuator body 12a, to puncture a vascular wall 32a. It may take only between approximately 100 milliseconds and two seconds for the microneedle to move from its furled state to its unfurled state.

The ends of the actuator at the retaining rings 22a and 22b remain rigidly fixed to the catheter 20. Thus, they do not deform during actuation. Since the actuator begins as a furled structure, its so-called pregnant shape exists as an unstable buckling mode. This instability, upon actuation, produces a large-scale motion of the microneedle approximately perpendicular to the central axis of the actuator body, causing a rapid puncture of the vascular wall without a large momentum transfer. As a result, a microscale opening is produced with very minimal damage to the surrounding tissue. Also, since the momentum transfer is relatively small, only a negligible bias force is required to hold the catheter and actuator in place during actuation and puncture.

The microneedle, in fact, travels so quickly and with such force that it can enter perivascular tissue 32b as well as vascular tissue. Additionally, since the actuator is “parked” or stopped prior to actuation, more precise placement and control over penetration of the vascular wall are obtained.

After actuation of the microneedle and delivery of the pharmaceutical to the target region via the microneedle, the activating fluid is exhausted from the open area 26 of the actuator, causing the expandable section 24 to return to its original, furled state. This also causes the microneedle to be withdrawn from the vascular wall. The microneedle, being withdrawn, is once again sheathed by the actuator.

As shown in FIG. 4A, the fabrication of the actuator 12 may start with a hollow tube or mandrel 36 that has a groove or slit 38 formed along part of its length. The tube or mandrel functions as a mold. It is coated with a dissolvable polymer that functions as a mold release device as discussed below. The wall thickness of the tube will define the cross-sectional dimension of the open area 26 of the actuator, and the exterior cross-sectional dimension of the tube will determine the exterior cross-sectional dimension of the actuator. The length of the tube, obviously, also determines the overall length of the actuator.

The retaining rings 22a and 22b are next placed at the opposite ends, respectively, of the tube (FIG. 4B). Specifically, they are slid over the exterior surface of the tube or into the interior surface of the tube. The tube and the retaining rings are then coated with a thin, rigid or semi-rigid, expandable material 40, such as Parylene, silicone, polyurethane or polyimide.

For instance, a Parylene C polymer may be gas vapor deposited onto and into the mold. Parylene is the trade name for the polymer poly-para-xylylene. Parylene C is the same monomer modified by the substitution of a chlorine atom for one of the aromatic hydrogens. Parylene C is used because of its conformality during deposition and its relatively high deposition rate, around 5 μm per hour.

The Parylene process is a conformal vapor deposition that takes place at room temperature. A solid dimer is first vaporized at about 150° C. and then cleaved into a monomer at about 650° C. This vaporized monomer is then brought into a room temperature deposition chamber, such as one available from Specialty Coating Systems of Indianapolis, IN, where it condenses and polymerizes onto the mold. Because the mean free path of the monomer gas molecules is on the order of 0.1 centimeter (cm), the Parylene deposition is very conformal. The Parylene coating is pinhole free at below a 25 nanometer (nm) thickness.

Due to the extreme conformality of the deposition process, Parylene will coat both the inside (via the slit 38) and outside of the mold. The Parylene coating inside and outside the mold may be on the order of 5 to 50 μm thick, and more typically about 25 μm thick.

Other Parylenes, such as Types N and D, may be used in place of Parylene C. The important thing is that the polymer be conformally deposited. That is, the deposited polymer has a substantially constant thickness regardless of surface topologies or geometries.

Additionally, a fluid flood and air purge process could be used to form a conformal polymer layer on and in the mold. Also, a dip-coating process could be used to form a conformal polymer layer on and in the mold. Polymers that may be used in this process include polyurethane, an epoxy or a silicone.

As shown in FIG. 4C, the next step is to release the actuator structure 12 from the mold or tube 36. This is accomplished by virtue of the mold release. Specifically, the dissolvable polymer that was initially coated onto the tube is dissolved in a solvent to release the actuator structure from the mold. The actuator structure is then opened for placement of the microneedle 14 on the surface 24a of the expandable section 24 of the actuator (see FIG. 4D). Alternatively, if the expandable section 24 and the microneedle 14 are both made of Parylene, then the microneedle may be molded directly into surface 24a. A technique for such direct molding is described in the above-identified application Ser. No. 09/877,653, which has been incorporated herein by reference. Also, at this point, a suitable opening or passageway may be formed at the proximal end of the actuator for establishing fluid communication between the open area 26 of the actuator and the delivery conduit 28.

The microneedle is then placed in fluid communication with the proximal end of the actuator by means of, for instance, the pharmaceutical supply tube 14d (FIG. 4E). The microneedle and supply tube may be joined together by a butt-weld, an ultra-sonic weld or an adhesive such as an epoxy. The microneedle 14 is then adhered to surface 24a by, for example, the metallic mesh-like structure 30 described above. (FIG. 4F)

Next, as shown in FIG. 4G, the retaining ring 22b of the actuator is joined to the lead end of the catheter 20 by, for example, and as discussed, a butt-weld, an ultra sonic weld or an adhesive such as an epoxy. The tip end of the catheter is joined to the retaining ring 22a in a similar fashion or during actuator fabrication. At this point, the appropriate fluid interconnects can be made between the lead end of the catheter, and the distal tip of the microneedle and the open area 26 of the actuator.

Various microfabricated devices can be integrated into the needle, actuator and catheter for metering flows, capturing samples of biological tissue, and measuring pH. The device 10, for instance, could include electrical sensors for measuring the flow through the microneedle as well as the pH of the pharmaceutical being deployed. The device 10 could also include an intravascular ultrasonic sensor (IVUS) for locating vessel walls, and fiber optics, as is well known in the art, for viewing the target region. For such complete systems, high integrity electrical, mechanical and fluid connections are provided to transfer power, energy, and pharmaceuticals or biological agents with reliability.

By way of example, the microneedle may have an overall length of between about 200 and 3,000 microns (μm). The interior cross-sectional dimension of the shaft 14b and supply tube 14d may be on the order of 20 to 250 μm, while the tube's and shaft's exterior cross-sectional dimension may be between about 100 and 500 μm. The overall length of the actuator body may be between about 5 and 50 millimeters (mm), while the exterior and interior cross-sectional dimensions of the actuator body can be between about 0.4 and 4 mm, and 0.5 and 5 mm, respectively. The gap or slit through which the central section of the actuator unfurls may have a length of about 4-40 mm, and a cross-sectional dimension of about 50-500 μm. The diameter of the delivery tube for the activating fluid may be about 100 μm. The catheter size may be between 1.5 and 15 French (Fr).

Variations of the invention include a multiple-buckling actuator with a single supply tube for the activating fluid. The multiple-buckling actuator includes multiple needles that can be inserted into or through a vessel wall for providing injection at different locations or times.

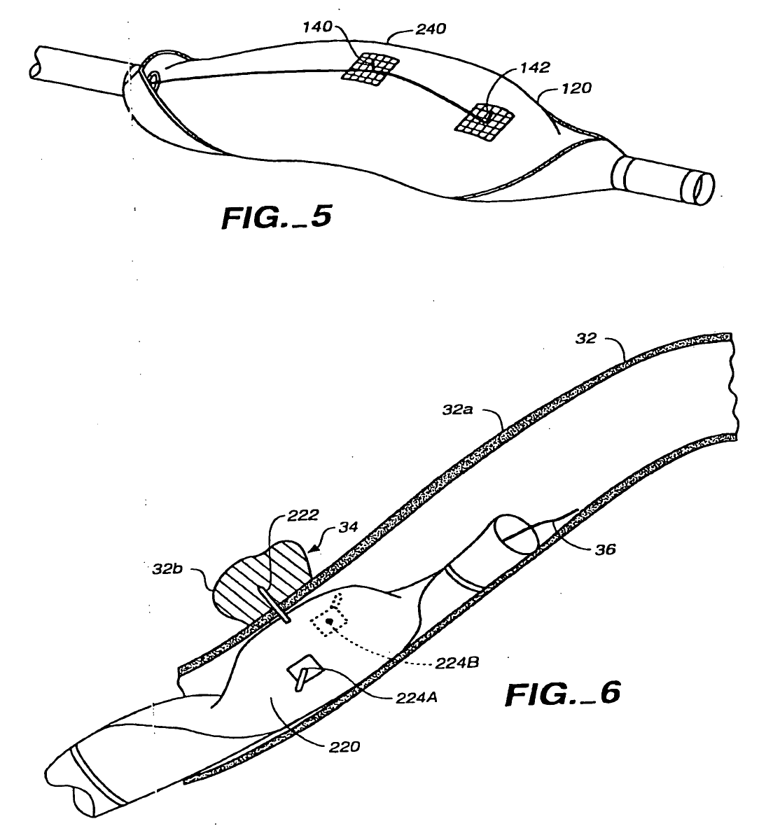

For instance, as shown in FIG. 5, the actuator 120 includes microneedles 140 and 142 located at different points along a length or longitudinal dimension of the central, expandable section 240. The operating pressure of the activating fluid is selected so that the microneedles move at the same time. Alternatively, the pressure of the activating fluid may be selected so that the microneedle 140 moves before the microneedle 142.

Specifically, the microneedle 140 is located at a portion of the expandable section 240 (lower activation pressure) that, for the same activating fluid pressure, will buckle outwardly before that portion of the expandable section (higher activation pressure) where the microneedle 142 is located. Thus, for example, if the operating pressure of the activating fluid within the open area of the expandable section 240 is two pounds per square inch (psi), the microneedle 140 will move before the microneedle 142. It is only when the operating pressure is increased to four psi, for instance, that the microneedle 142 will move. Thus, this mode of operation provides staged buckling with the microneedle 140 moving at time t₁, and pressure p₁, and the microneedle 142 moving at time t₂ and p₂, with t₁, and p₁, being less than t₂ and p₂, respectively.

This sort of staged buckling can also be provided with different pneumatic or hydraulic connections at different parts of the central section 240 in which each part includes an individual microneedle.

Also, as shown in FIG. 6, an actuator 220 could be constructed such that its needles 222 and 224A move in different directions. As shown, upon actuation, the needles move at angle of approximately 90° to each other to puncture different parts of a vessel wall. A needle 224B (as shown in phantom) could alternatively be arranged to move at angle of about 180° to the needle 224A.

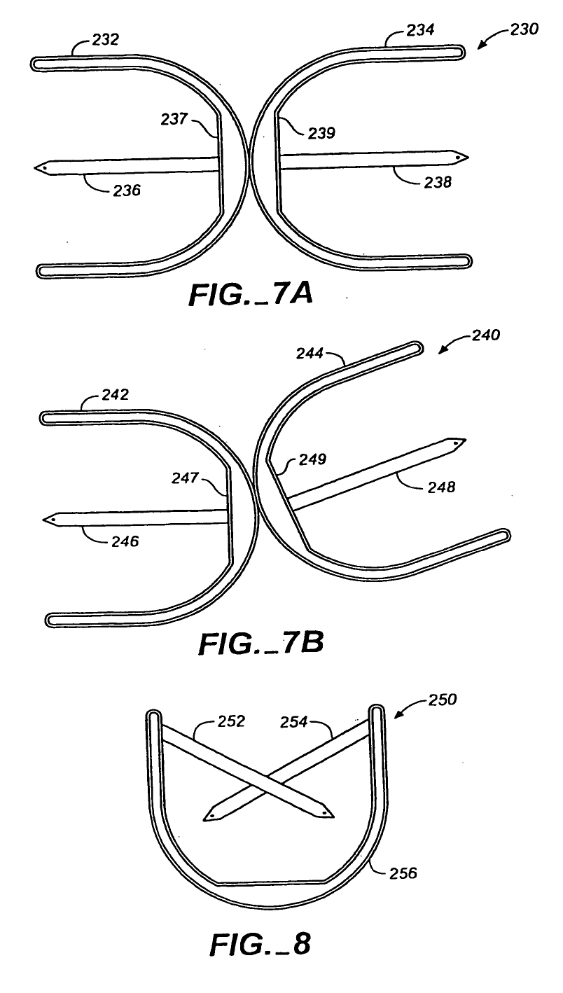

Moreover, as shown in FIG. 7A, in another embodiment, an actuator 230 comprises actuator bodies 232 and 234 including needles 236 and 238, respectively, that move approximately horizontally at angle of about 180° to each other. Also, as shown in FIG. 7B, an actuator 240 comprises actuator bodies 242 and 244 including needles 242 and 244, respectively, that are configured to move at some angle relative to each other than 90° or 180°. The central expandable section of the actuator 230 is provided by central expandable sections 237 and 239 of the actuator bodies 232 and 234, respectively. Similarly, the central expandable section of the actuator 240 is provided by central expandable sections 247 and 249 of the actuator bodies 242 and 244, respectively.

Additionally, as shown in FIG. 8, an actuator 250 may be constructed that includes multiple needles 252 and 254 that move in different directions when the actuator is caused to change from the unactuated to the actuated condition. The needles 252 and 254, upon activation, do not move in a substantially perpendicular direction relative to the longitudinal axis of the actuator body 256.

Damage to the inside of arteries caused by abrasion or lesion can seriously affect patients with sometimes drastic consequences such as vasospasm, leading to arterial collapse and loss of blood flow. Breach of the arterial wall through interventional surgical needles can prevent such problems.

Intravascular catheters described above may be used to deliver estrogens according to the present invention to patients at risk of hyperplasia or suffering vulnerable plaque. Patients at risk of hyperplasia will usually be those having arteriosclerotic disease which has previously been treated by balloon angioplasty, atherectomy, stenting, or other primary interventional technique which may injure the blood vessel wall. It is known that such injury can initiate smooth muscle cell migration, leading to neointimal hyperplasia. Direct injection of estrogens according to the present invention into a perivascular space or directly into the adventitia will inhibit or prevent such hyperplasia. Direct injection of the estrogens may also be used for the primary treatment of vulnerable plaques. Vulnerable plaques are well described in medical literature and have been found to be at particular risk of rupture and are therefore of particular concern to the patient. Thus, the methods of the present invention can be used to inject estrogen into or adjacent to regions of vulnerable plaque in patients diagnosed of having such plaque. Particular methods for diagnosing patents with vulnerable plaque are described, for example, in U.S. Pat. Nos. 6,475,159; 6,450,971; 6,245,026; and 5,924,997, the full disclosures of which are incorporated herein by reference.

A number of embodiments of the invention have been described. Nevertheless, it will be understood that various modifications may be made without departing from the spirit and scope of the invention as claimed hereinafter.

What is claimed is:

1. A method for treating a blood vessel, said method comprising injecting an estrogen into a location beyond the endothelium of the blood vessel.

2. A method as in claim 1, wherein the blood vessel is an artery.

3. A method as in claim 2, wherein the artery is a coronary artery.

4. A method as in claim 1, wherein the estrogen is an estradiol.

5. A method as in claim 4, wherein the estradiol is selected from the group consisting of 17-beta-estradiol and estradiol cypionate.

6. A method as in claim 1, wherein the blood vessel is at risk of hyperplasia.

7. A method as in claim 6, wherein the estrogen is injected proximate the region of hyperplasia risk.

8. A method as in claim 1, wherein the blood vessel has regions of vulnerable plaque.

9. A method as in claim 8, wherein the estrogen is injected proximate a region of vulnerable plaque.

10. A method as in claim 1, wherein injecting comprises introducing a catheter into a lumen of the blood vessel and advancing a needle from the catheter, wherein the estrogen is injected through the needle.

11. A method as in claim 10, wherein the needle is advanced into a perivascular space beyond the outside of the endothelium.

12. A method as in claim 11, wherein the needle is advanced into the adventia surrounding the blood vessel.

13. A method as in claim 1, wherein the estrogen is injected in an amount sufficient to permeate circumferentially around the endothelium and into the adventia over an axial length of at least 1 cm.

14. A method as in claim 1, wherein the needle is advanced in a radial direction to a depth in the adventitia equal to at least 10% of the mean luminal diameter at the blood vessel location.

15. A method as in claim 14, wherein the depth is a distance in the range from 10% to 50% of the mean luminal diameter.

16. A system for treating a blood vessel, said system comprising:
an amount of an estrogen, and
an intravascular catheter having a needle for injecting the estrogen into a location beyond the endothelium of the blood vessel.
